(19) United States
(12) Patent Application Publication
(10) Pub. No.: US 2014/0194761 A1
(43) Pub. Date: Jul. 10, 2014

(54) METHOD AND APPARATUS FOR MONITORING BIOSIGNAL WITH LOW POWER CONSUMPTION

(71) Applicant: Samsung Electronics Co., Ltd., Suwon-si (KR)

(72) Inventors: Tak Hyung Lee, Seoul (KR); Kun Kook Park, Suwon-si (KR); Kun Soo Shin, Seongnam-si (KR)

(73) Assignee: Samsung Electronics Co., Ltd., Suwon-si (KR)

(21) Appl. No.: 14/150,396

(22) Filed: Jan. 8, 2014

(30) Foreign Application Priority Data
Jan. 8, 2013 (KR) 10-2013-0001914

(51) Int. Cl. A61B 5/04

(52) U.S. Cl. CPC A61B 5/04012
USPC 600/509; 600/547; 600/544; 600/546

(57) ABSTRACT

A method of monitoring a biosignal includes receiving a biosignal and a reference signal, determining a signal state of the biosignal based on the reference signal, and analyzing the biosignal using an analysis technique having a resource consumption depending on the signal state.

### CROSS-REFERENCE TO RELATED APPLICATIONS

This application claims the benefit under 35 USC 119(a) of Korean Patent Application No. 10-2013-0001914 filed on Jan. 8, 2013, in the Korean Intellectual Property Office, the disclosure of which is incorporated herein by reference in its entirety for all purposes.

### BACKGROUND

1. Field

The following description relates to a method and an apparatus for monitoring a biosignal, such as an electrocardiogram (ECG) signal, with low power consumption based on a signal state of a signal related to a motion of a user.

2. Description of Related Art

An electrocardiogram (ECG) measurement device is used for heart disease management, health and fitness measurement, and other purposes. Among various types of ECG measurement devices being marketed, usage of a portable ECG measurement device is increasing due to its convenience of use. In an effort to meet demands for miniaturization, a size of portable ECG measurement device is being reduced. However, a downside of this trend is a limited battery capacity, and as a consequence, a length of time the device can be used continuously may be reduced.

### SUMMARY

In one general aspect, a method of monitoring a biosignal includes receiving a biosignal and a reference signal; determining a signal state of the biosignal based on the reference signal; and analyzing the biosignal using an analysis technique having a resource consumption depending on the signal state.

The determining of the signal state of the biosignal may include determining a variable based on the reference signal; and determining the signal state of the biosignal based on the variable.

The determining of the variable may include determining the variable based on the reference signal in a resting period.

The analyzing of the biosignal may include reducing a motion artifact in response to the signal state indicating that the biosignal may include a motion artifact; and analyzing the biosignal in which the motion artifact has been reduced using a motion artifact-specialized analysis.

The analyzing of the biosignal may include analyzing the biosignal using a software analysis in response to the signal state being an unstable signal state; and analyzing the biosignal using a combination analysis in response to the signal state being a stable state.

The analyzing of the biosignal using the combination analysis may include detecting a feature point of the biosignal using an analog circuit or a digital chip; and analyzing the biosignal based on the feature point.

The detecting of the feature point of the biosignal may include detecting the feature point of the biosignal based on a threshold using the analog circuit or the digital chip, the threshold being determined based on the biosignal or the reference signal.

The analyzing of the biosignal using the software analysis may include extracting a feature point of the biosignal by performing a digital signal processing (DSP) operation on the biosignal using a microcontroller; and analyzing the biosignal based on the feature point.

In another general aspect, a non-transitory computer-readable storage medium stores a program for controlling a computer to perform the method described above.

In another general aspect, an apparatus for monitoring a biosignal includes a receiver configured to receive a biosignal and a reference signal; a signal state determiner configured to determine a signal state of the biosignal based on the reference signal; and a signal analyzer configured to analyze the biosignal using an analysis technique having a resource consumption depending on the signal state.

The signal analyzer may include a microcontroller configured to perform a digital signal processing (DSP) operation on the biosignal to analyze the biosignal in response to the signal state being an unstable signal state.

The signal analyzer may include a comparator configured to compare a characteristic of the biosignal with a threshold in response to the signal state being a stable signal state to determine whether the characteristic of the biosignal exceeds the threshold, the threshold being determined based on the biosignal or the reference signal; a pulse generator configured to generate a pulse each time the comparator determines that the characteristic of the biosignal exceeds the threshold; and a heart rate calculator configured to calculate a heart rate by measuring a pulse interval of pulses generated by the pulse generator, and calculating the heart rate based on the pulse interval.

The threshold may be a threshold for an amplitude of the biosignal, or a threshold for a root mean square (RMS) of the biosignal, or a threshold for a feature point of the biosignal.

The receiver may include a signal measurer configured to measure the biosignal and the reference signal.

The receiver may include a signal measurer configured to measure the biosignal; and a communicator configured to receive the reference signal from an external device configured to measure the reference signal.

The reference signal may be an electrocardiogram (ECG) signal, or an accelerometer signal, or a half-cell potential (HCP) signal, or an impedance signal, or a lightwave signal, or a strain gauge signal, or a signal generated by a motion of a user.

The biosignal may be an ECG signal, or an electroencephalogram (EEG) signal, or an electrooculogram (EOG) signal, or an electromyogram (EMG) signal.

The receiver may include a signal measurer configured to be attached to a body of a user to measure the biosignal; and the signal state of the biosignal is influenced by a motion state of a user or a location of the signal measurer on the body of the user.

In another general aspect, a method of monitoring a biosignal includes selecting one of a plurality of analysis techniques having different resource consumptions based on a biosignal; and analyzing the biosignal using the selected analysis technique.

The plurality of analysis techniques may respectively correspond to different stabilities of the biosignal.

The selecting may include selecting one of the plurality of analysis techniques having different resource consumptions based on a stability of the biosignal.

A resource consumption of the selected analysis technique may decrease as the stability of the biosignal increases.

The biosignal may be a biosignal of a user, and may be influenced by a motion of the user; and the selecting may include selecting one of the plurality of analysis techniques having different resource consumptions based on a characteristic of the biosignal indicative of the motion of the user.

A resource consumption of the selected analysis technique may decrease as the motion of the user decreases.

The method of claim may further include obtaining a reference signal indicative of the motion of the user; and determining the characteristic of the biosignal based on the reference signal.

Other features and aspects will be apparent from the following detailed description, the drawings, and the claims.

### BRIEF DESCRIPTION OF THE DRAWINGS

FIG. 1 is a schematic diagram illustrating an example of an apparatus for monitoring a biosignal with low power consumption.

FIG. 2 is a flowchart illustrating an example of a method of monitoring a biosignal with low power consumption.

FIG. 3A is a graph illustrating an example of a biosignal measured during a resting period.

FIG. 3B is a graph illustrating an example of a biosignal measured after a motion occurs.

FIG. 4A is a graph illustrating an example of a measured half-cell potential (HCP) signal as an example of a reference signal.

FIG. 4B is a graph illustrating an example of a measured electrocardiogram (ECG) signal as an example of a biosignal.

FIG. 5A is a graph illustrating an example of an HCP signal as an example of a reference signal measured after a motion occurs.

FIG. 5B is a graph illustrating an example of an ECG signal as an example of a biosignal measured after a motion occurs.

FIG. 6A is a flowchart illustrating an example of biosignal analysis based on an analysis scheme.

FIG. 6B is a flowchart illustrating an example of biosignal analysis based on an analysis algorithm.

FIG. 7A is a flowchart illustrating an example of biosignal analysis using a combination analysis scheme.

FIG. 7B is a graph illustrating an example of biosignal analysis using a combination analysis scheme.

FIG. 8 is a block diagram illustrating an example of an apparatus for monitoring a biosignal with low power consumption.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples, and are not limited to those set forth herein and may be changed as will be apparent to one of ordinary skill in the art, with the exception of operations necessarily occurring in a certain order. Also, description of functions and constructions that are well known to one of ordinary skill in the art may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

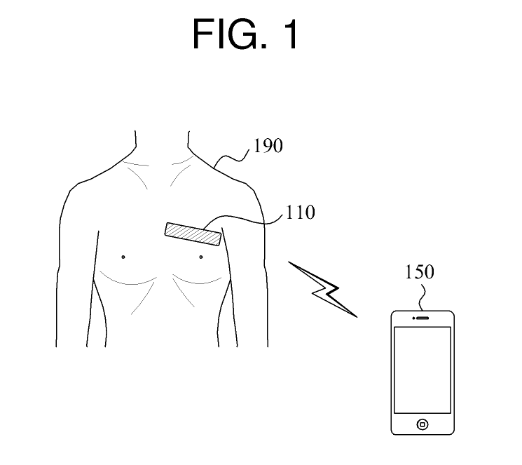

FIG. 1 is a schematic diagram illustrating an example of an apparatus 110 for monitoring a biosignal with low power consumption. Referring to FIG. 1, the apparatus 110 for monitoring a biosignal with low power consumption measures a biosignal from a user 190, and analyzes the measured biosignal. The apparatus 110 determines a signal state of the measured biosignal based on a reference signal, and analyzes the biosignal using a low resource consumption approach based on the determined signal state. In other words, the apparatus 110 analyzes the biosignal using an analysis technique selected based on the determined signal state to minimize the consumption of resources, or analyzes the biosignal using an analysis technique having a resource consumption depending on the determined signal state. The analysis technique may be an analysis scheme, such as a hardware analysis scheme, a software analysis scheme, or a combination analysis scheme that is a combination of a hardware analysis scheme and a software analysis scheme as will be described in greater detail below. Alternatively, the analysis technique may be an analysis algorithm, such as an analysis algorithm based on a threshold, a general analysis algorithm that is more complex than the analysis algorithm based on a threshold, or a motion artifact-specialized analysis algorithm.

The reference signal is used in determining an analysis technique, and may be measured by an external device 150. The measured reference signal may be transmitted to the apparatus 110 for monitoring a biosignal with low power consumption via a wired or wireless connection.

For example, an electrocardiogram (ECG) measurement device may measure an ECG signal, and may convert the ECG signal into a digital signal. A feature point in the digital ECG signal may be detected for an analysis of the ECG signal to be performed. A central processing unit (CPU) or a digital signal processor (DSP) embedded in the ECG measurement device may be used, or a device with an ECG analysis function may be used via a wired or wireless connection. An ECG feature point analysis algorithm may run continuously while the ECG signal is being measured, which may have a significant effect on the power consumption of the ECG measurement device.

The ECG feature point analysis algorithm may involve, for example, R-peak detection for a heart rate analysis. Since an ECG signal frequently includes noise, the ECG feature point analysis algorithm is designed to compensate for noise in the ECG signal to ensure accurate feature point analysis of the ECG signal. This requires complex computations to compensate for the noise, resulting in an increase in power consumption. However, these complex computations are unnecessary when the ECG signal does not include noise, such as, for example, when a user is at rest.

Accordingly, the power consumption of the ECG measurement device attributable to the ECG feature point analysis algorithm may be minimized by selecting an appropriate ECG analysis technique based on a signal state of an ECG signal when the ECG signal is measured.

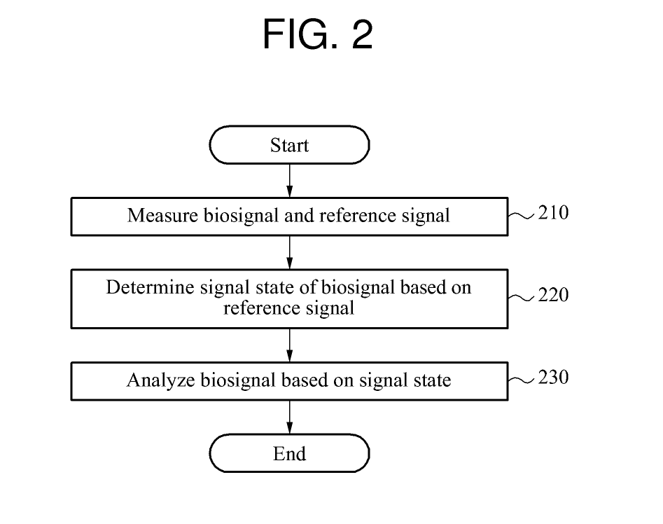

FIG. 2 is a flowchart illustrating an example of a method of monitoring a biosignal with low power consumption. One factor limiting use of a biosignal measurement device in a mobile environment a length of time the device can be used continuously. To increase the length of time the device can be used continuously, reducing power consumption is a major factor. For example, in the case of an ECG measurement device, when a heart rate analysis of an ECG signal is performed with a low power consumption, the overall power consumption of the ECG measurement device may be reduced. As a result, the length of time the ECG measurement device can be used continuously may be increased.

In 210, a biosignal is measured. The biosignal may be an ECG signal, an electroencephalogram (EEG) signal, an electrooculogram (EOG) signal, an electromyogram (EMG) signal, or any other biosignal. In addition to the biosignal, a reference signal is measured. The reference signal is used in determining a signal state of the biosignal, and may be an ECG signal, an accelerometer signal, a half-cell potential (HCP) signal, an impedance signal, a lightwave signal, a strain gauge signal, a signal generated by a motion of a user, or any other signal that can be used to determine the signal state of the biosignal.

In 220, the signal state of the biosignal is determined based on the reference signal. The signal state may be influenced by a motion state of a user or a location of a biosignal measurement device on a body of the user. For example, the signal state may be the motion state of the user.

In one example, a variable for determining the signal state may be determined. The variable may be an amplitude of the reference signal, a root mean square (RMS) of the reference signal, or a feature point of the biosignal, for example, an R-peak of an ECG signal. The variable may be determined based on a reference signal in a resting period. The resting period may be a period in which a user is at rest, i.e., not moving. The signal state of the biosignal, such as the motion state of the user, may be determined based on the determined variable.

In 230, the biosignal is analyzed using a low resource consumption approach based on the determined signal state. The signal state may be influenced by a motion of the user, or an external factor influencing a waveform of the biosignal.

In one example, when the biosignal is determined to be a biosignal in a resting period, the biosignal may be analyzed using an analysis algorithm requiring a small amount of computation and having a low power consumption. When the biosignal is determined to include a motion artifact, the biosignal may be processed to reduce the motion artifact, and the biosignal having the reduced motion artifact may be analyzed using a motion artifact-specialized analysis algorithm. The motion artifact may be reduced by a motion artifact reduction function during signal processing.

In another example, when the signal state of the biosignal is determined to be unstable based on the reference signal, the biosignal may be analyzed using a software analysis scheme, and when the signal state of the biosignal is determined to be stable based on the reference signal, the biosignal may be analyzed using a combination analysis scheme.

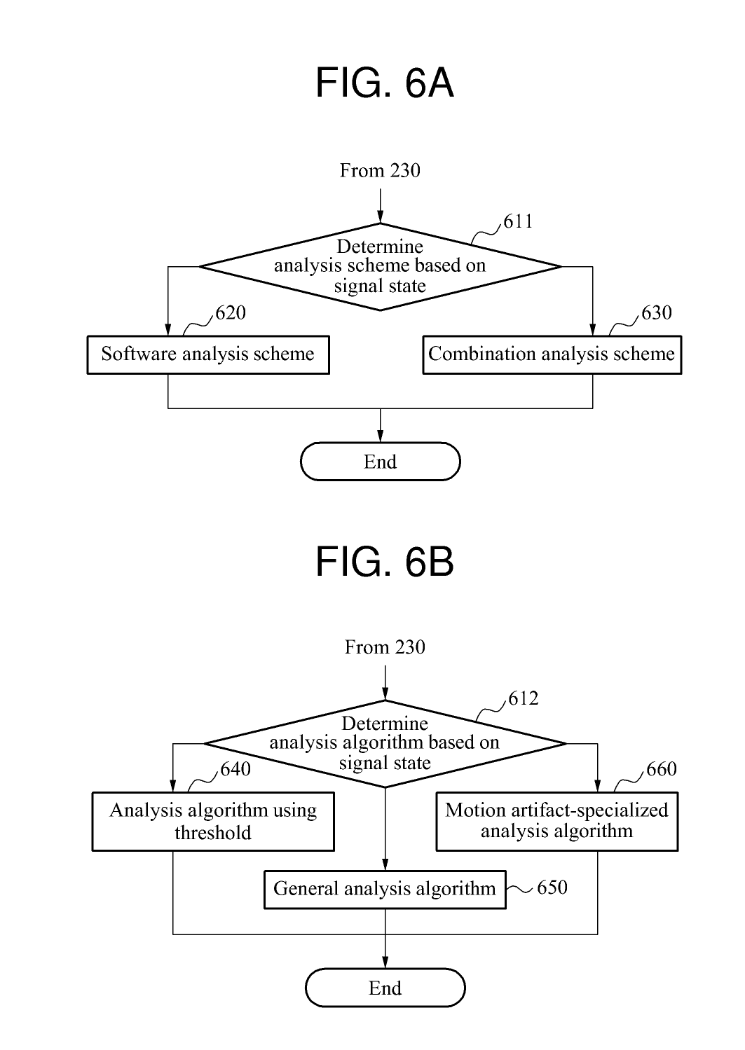

Accordingly, the length of time the biosignal measurement device can be used continuously may be prolonged by the method of monitoring a biosignal with low power consumption. A more detailed description of the software analysis scheme and the combination analysis scheme is provided below with reference to FIG. 6A.

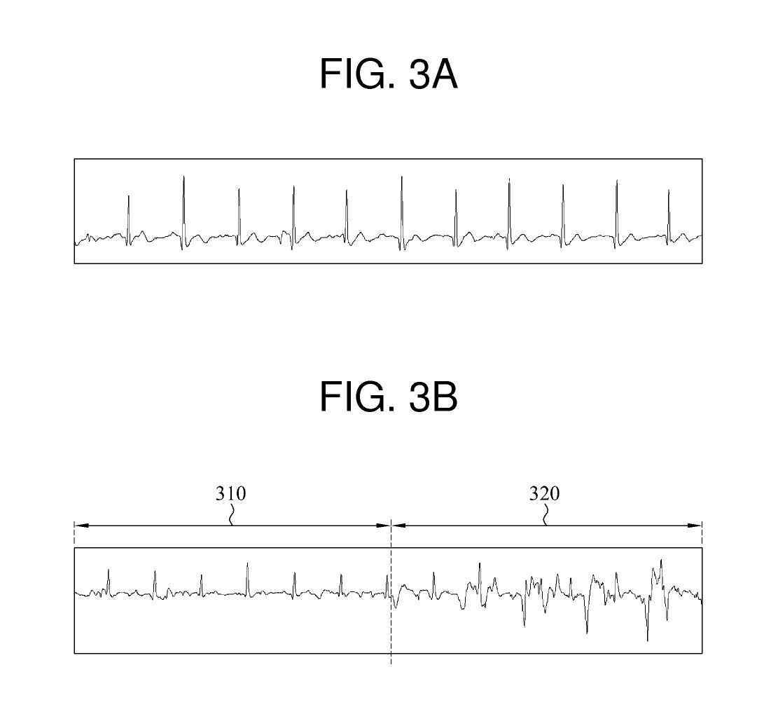

FIG. 3A is a graph illustrating an example of a biosignal measured during a resting period, and FIG. 3B is a graph illustrating an example of a biosignal measured after a motion occurs. In these examples, the biosignal is an ECG signal, but the biosignal may be any biosignal.

In FIG. 3A, each feature point of a QPRS wave in an ECG signal in a resting period is shown. In FIG. 3B, an ECG signal 310 in a resting period and an ECG signal 320 generated after a motion occurs are shown. The ECG signal 320 generated after the motion occurs may be difficult to measure, or it may be difficult to extract each feature point of the QPRS wave accurately.

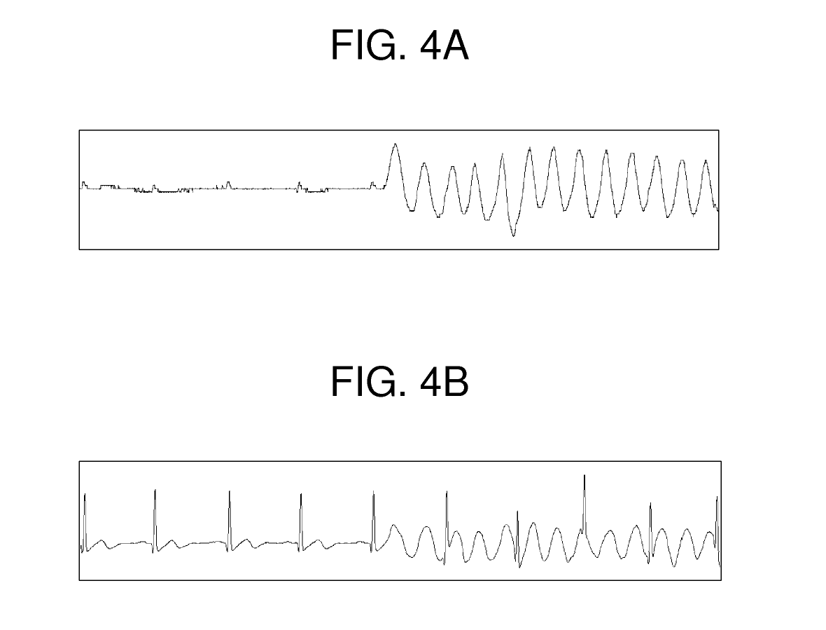

FIG. 4A is a graph illustrating an example of a measured HCP signal as an example of a reference signal, and FIG. 4B is a graph illustrating an example of a measured ECG signal as an example of a biosignal. The reference signal in FIG. 4A and the biosignal in FIG. 4B may be measured in operation 210 of FIG. 2.

As described above, a reference signal may be used in determining a motion state or determining a variable for use in determining an analysis algorithm. For example, the reference signal may be an ECG signal, an accelerometer signal, an HCP signal, an impedance signal, a lightwave signal, a strain gauge signal, or a signal generated by a motion of a user. The reference signal may be acquired from an external device, or from a biosignal measurement device mounted in the apparatus for monitoring a biosignal with low power consumption.

In FIG. 4A, a state change of an HCP signal as an example of a reference signal when a motion occurs after a resting period is shown. In FIG. 4B, a state change of an ECG signal as an example of a biosignal when a motion occurs after a resting period is shown. Since the signal of FIG. 4A has a more regular pattern than the signal of FIG. 4B, performing signal analysis of the reference signal of FIG. 4A may be easier than performing signal analysis of the biosignal of FIG. 4B.

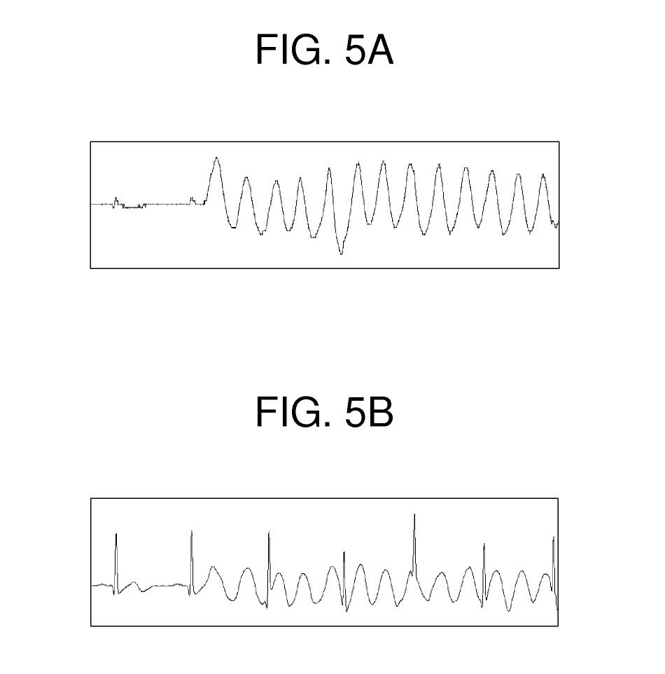

FIG. 5A is a graph illustrating an example of an HCP signal as an example of a reference signal measured after a motion occurs, and FIG. 5B is a graph illustrating an example of an ECG signal as an example of a biosignal measured after a motion occurs. The signal state of the biosignal in FIG. 5B may be determined based on the reference signal in FIG. 5A in operation 220 in FIG. 2.

After the biosignal and the reference signal are measured, a variable for use in determining the motion state may be determined by performing an algorithm for determining the variable. For example, the variable may be a threshold for an amplitude of the reference signal, a threshold for an RMS of the reference signal, a threshold for a feature point, or other variable. The variable may be optimized by an individual user. The feature point may be, for example, an R-peak.

The apparatus for monitoring a biosignal with low power consumption may determine a variable in advance of being used, or may receive an input variable from a separate external device. The external device may be, for example, an accelerometer sensor of a mobile phone, a global positioning system (GPS), or other device capable of measuring a reference signal, determining a variable, and inputting the measured reference signal and the determined variable to the apparatus for monitoring a biosignal with low power consumption.

The apparatus for monitoring a biosignal with low power consumption may determine the signal state of the motion-affected biosignal based on the determined variable.

FIG. 6A is a flowchart illustrating an example of a biosignal analysis based on an analysis scheme, and FIG. 6B is a flowchart illustrating an example of biosignal analysis based on an analysis algorithm.

Referring to FIG. 6A, in 611, a biosignal analysis scheme is determined based on a signal state of the biosignal determined based on a reference signal.

In 620, when the signal state of the biosignal is determined to be unstable, the biosignal is analyzed using a software analysis scheme. A case in which the biosignal is unstable may include a case in which the user is in an active state, for example, in motion. For example, if each feature of the ECG signal is not measured, or a value of the accelerometer signal indicates that an acceleration exceeds a predetermined value, the signal state may be determined to be unstable. As another example, the signal state may be determined to be unstable based on the HCP signal changed by the motion of the user, or the lightwave signal, or the strain gauge signal. The software analysis scheme may analyze a biosignal by extracting a feature point from the biosignal by performing a digital signal processing (DSP) operation using a microcontroller. An algorithm for the DSP operation may be applied selectively based on the signal state of the biosignal.

In 630, when the signal state of the biosignal is determined to be stable, the biosignal is analyzed using a combination analysis scheme. A case in which the biosignal is stable may include a case in which the user is in a static state, for example, not in motion. The combination analysis scheme may be a combination of a software scheme and a hardware scheme, and may analyze a biosignal by detecting a feature point of the biosignal using, for example, an analog circuit or a digital chip as example of a hardware scheme, and analyzing the detected feature points by performing, for example, a DSP operation as an example of a software scheme. A threshold for detecting the feature point of the biosignal, for example, an R-peak of an ECG signal, may be determined based on the biosignal or the reference signal.

Referring to FIG. 6B, in 612, a biosignal analysis algorithm is determined based on a signal state of a biosignal, such as a motion state of a user, determined based on a reference signal.

The reference signal, for example, an accelerometer signal related to a motion of a user, may be measured simultaneously with the measuring the biosignal, for example, an ECG signal. A signal used for determining the motion of the user may be an RMS of an ECG signal, a value of an accelerometer signal, a value of an HCP signal, and a variable of other reference signals. In one example, as an amount of motion determined to be present in the biosignal based on the reference signal decreases, an algorithm with a lower power consumption may be used. A value used to determine the amount of motion may be changed. For example, the value may be variable according to the user, and personalized to the user.

In 640, when the biosignal, for example, an ECG signal, is determined to be stable due to no motion or relatively little motion, an analysis algorithm using a threshold, for example, an ECG feature point detection algorithm using a threshold, may be used. This algorithm may be implemented in a hardware device, and thereby enabling a DSP operation be minimized, thereby reducing a resource consumption of the apparatus for monitoring a biosignal with low power consumption. The resource may be an amount of computation, a power consumption, or any other resource consumed in analysis of the biosignal.

In 650, when an amount of motion is relatively increased, a general analysis algorithm that is more complex than the analysis algorithm using a threshold may be used. For example, the general analysis algorithm may analyze at least one of the features of an ECG signal, such as at least one of the features of the PQRST waves of the ECG signal.

In 660, when it is difficult to analyze the biosignal, for example, an ECG signal, using the general analysis algorithm due to a great amount of motion, a motion artifact reduction function may be performed on the biosignal, and the biosignal with a reduced motion artifact, for example, the ECG signal with a reduced motion artifact, may be analyzed. For example, a motion artifact-specialized analysis algorithm may be used.

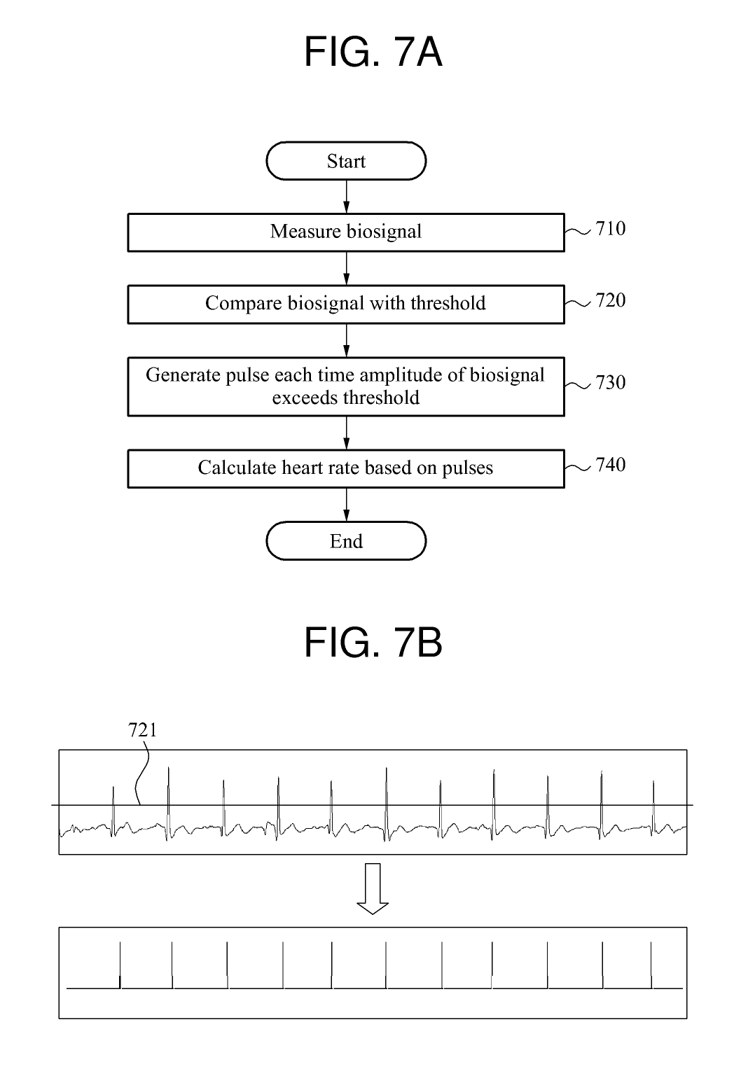

FIG. 7A is a flowchart illustrating an example of biosignal analysis using a combination analysis scheme, and FIG. 7B is a graph illustrating an example of biosignal analysis using a combination analysis scheme.

In 710, a biosignal is measured.

In 720, the measured biosignal is compared with a predetermined threshold 721 by a comparator. For example, the threshold 721 may be optimized and determined by an individual user, and may be determined to detect a feature point of the biosignal, in this case, an R-peak of an ECG signal.

In 730, a pulse is generated each time an amplitude of the measured biosignal exceeds the threshold 721.

In 740, a heart rate is calculated based on the generated pulses. The heart rate may be calculated by measuring a pulse interval of the generated pulses. In one example, the combination analysis scheme, namely, a combination of a hardware scheme and a software scheme, may generate pulses using a hardware analysis scheme during a resting period, and may measure a pulse interval of the generated pulses using a software analysis scheme. Accordingly, an amount of computation in the software analysis scheme may be reduced.

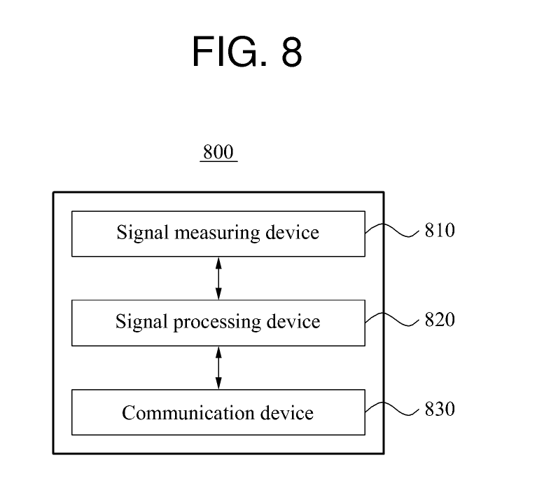

FIG. 8 is a block diagram illustrating an example of an apparatus 800 for monitoring a biosignal with low power consumption. Referring to FIG. 8, the apparatus 800 for monitoring a biosignal with low power consumption includes a signal measuring device 810, a signal processing device 820, and a communication device 830.

The signal measuring device 810 measures a biosignal, such as an ECG signal. Alternatively, the biosignal may be an EEG signal, an EOG signal, or an EMG signal.

The signal processing device 820 includes a signal state determination device and a signal analysis device. The signal state determination device determines a signal state of the biosignal based on a reference signal, and the signal analysis device analyzes the reference signal using a low resource consumption approach based on the determined signal state.

The signal analysis device may include a microcontroller to perform a DSP operation for analysis of the biosignal when the signal state is unstable, a comparator to compare the biosignal with a threshold determined based on the biosignal or the reference signal when the signal state is stable, a pulse generator to generate a pulse each time an amplitude of the biosignal exceeds the threshold, and a heart rate calculator to calculate a heart rate by measuring a pulse interval of the generated pulses. The threshold used in the comparator may be, for example, a threshold for an amplitude of the biosignal, a threshold for an RMS of the biosignal, or a threshold for a feature point. The feature point may be, for example, an R-peak.

The communication device 830 transmits the biosignal analysis result to another device via a wired or wireless connection. Also, the communication device 830 may receive any one or any combination of a reference signal measured by an external device, a variable to be used in determining a signal state determined by the external device, and a result of a reference signal analyzed by the external device.

The apparatus 800 for monitoring a biosignal with low power consumption may include a non-transitory computer-readable storage medium storing one or more programs for performing the method for monitoring the biosignal with low power consumption. The method may include selecting an analysis scheme having a resource consumption depending on a signal state of the biosignal, and applying the selected analysis scheme to the biosignal.

The apparatus 110 for monitoring a biosignal with low power consumption and the external device 150 illustrated in FIG. 1 and the apparatus 800 for monitoring a biosignal with low power consumption, the signal measuring device 810, the signal processing device 820, and the communication device 830 illustrated in FIG. 8 that perform the various operations illustrated in FIGS. 2, 6A, 6B, 7A, and 7B may be implemented using one or more hardware components, one or more software components, or a combination of one or more hardware components and one or more software components.

A hardware component may be, for example, a physical device that physically performs one or more operations, but is not limited thereto. Examples of hardware components include resistors, capacitors, inductors, power supplies, frequency generators, operational amplifiers, power amplifiers, low-pass filters, high-pass filters, band-pass filters, analog-to-digital converters, digital-to-analog converters, and processing devices.

A software component may be implemented, for example, by a processing device controlled by software or instructions to perform one or more operations, but is not limited thereto. A computer, controller, or other control device may cause the processing device to run the software or execute the instructions. One software component may be implemented by one processing device, or two or more software components may be implemented by one processing device, or one software component may be implemented by two or more processing devices, or two or more software components may be implemented by two or more processing devices.

A processing device may be implemented using one or more general-purpose or special-purpose computers, such as, for example, a processor, a controller and an arithmetic logic unit, a digital signal processor, a microcomputer, a field-programmable array, a programmable logic unit, a microprocessor, or any other device capable of running software or executing instructions. The processing device may run an operating system (OS), and may run one or more software applications that operate under the OS. The processing device may access, store, manipulate, process, and create data when running the software or executing the instructions. For simplicity, the singular term “processing device” may be used in the description, but one of ordinary skill in the art will appreciate that a processing device may include multiple processing elements and multiple types of processing elements. For example, a processing device may include one or more processors, or one or more processors and one or more controllers. In addition, different processing configurations are possible, such as parallel processors or multi-core processors.

A processing device configured to implement a software component to perform an operation A may include a processor programmed to run software or execute instructions to control the processor to perform operation A. In addition, a processing device configured to implement a software component to perform an operation A, an operation B, and an operation C may have various configurations, such as, for example, a processor configured to implement a software component to perform operations A, B, and C; a first processor configured to implement a software component to perform operation A, and a second processor configured to implement a software component to perform operations B and C; a first processor configured to implement a software component to perform operations A and B, and a second processor configured to implement a software component to perform operation C; a first processor configured to implement a software component to perform operation A, a second processor configured to implement a software component to perform operation B, and a third processor configured to implement a software component to perform operation C; a first processor configured to implement a software component to perform operations A, B, and C, and a second processor configured to implement a software component to perform operations A, B, and C, or any other configuration of one or more processors each implementing one or more of operations A, B, and C. Although these examples refer to three operations A, B, C, the number of operations that may implemented is not limited to three, but may be any number of operations required to achieve a desired result or perform a desired task.

Software or instructions for controlling a processing device to implement a software component may include a computer program, a piece of code, an instruction, or some combination thereof, for independently or collectively instructing or configuring the processing device to perform one or more desired operations. The software or instructions may include machine code that may be directly executed by the processing device, such as machine code produced by a compiler, and/or higher-level code that may be executed by the processing device using an interpreter. The software or instructions and any associated data, data files, and data structures may be embodied permanently or temporarily in any type of machine, component, physical or virtual equipment, computer storage medium or device, or a propagated signal wave capable of providing instructions or data to or being interpreted by the processing device. The software or instructions and any associated data, data files, and data structures also may be distributed over network-coupled computer systems so that the software or instructions and any associated data, data files, and data structures are stored and executed in a distributed fashion.

For example, the software or instructions and any associated data, data files, and data structures may be recorded, stored, or fixed in one or more non-transitory computer-readable storage media. A non-transitory computer-readable storage medium may be any data storage device that is capable of storing the software or instructions and any associated data, data files, and data structures so that they can be read by a computer system or processing device. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, or any other non-transitory computer-readable storage medium known to one of ordinary skill in the art.

Functional programs, codes, and code segments for implementing the examples disclosed herein can be easily constructed by a programmer skilled in the art to which the examples pertain based on the drawings and their corresponding descriptions as provided herein.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of monitoring a biosignal, the method comprising:
receiving a biosignal and a reference signal;
determining a signal state of the biosignal based on the reference signal; and
analyzing the biosignal using an analysis technique having a resource consumption depending on the signal state.

2. The method of claim 1, wherein the determining of the signal state of the biosignal comprises:
determining a variable based on the reference signal; and
determining the signal state of the biosignal based on the variable.

3. The method of claim 2, wherein the determining of the variable comprises determining the variable based on the reference signal in a resting period.

4. The method of claim 1, wherein the analyzing of the biosignal comprises:
reducing a motion artifact in response to the signal state indicating that the biosignal includes a motion artifact; and
analyzing the biosignal in which the motion artifact has been reduced using a motion artifact-specialized analysis.

5. The method of claim 1, wherein the analyzing of the biosignal comprises:
analyzing the biosignal using a software analysis in response to the signal state being an unstable signal state; and
analyzing the biosignal using a combination analysis in response to the signal state being a stable state.

6. The method of claim 5, wherein the analyzing of the biosignal using the combination analysis comprises:
detecting a feature point of the biosignal using an analog circuit or a digital chip; and
analyzing the biosignal based on the feature point.

7. The method of claim 6, wherein the detecting of the feature point of the biosignal comprises detecting the feature point of the biosignal based on a threshold using the analog circuit or the digital chip, the threshold being determined based on the biosignal or the reference signal.

8. The method of claim 5, wherein the analyzing of the biosignal using the software analysis comprises:
extracting a feature point of the biosignal by performing a digital signal processing (DSP) operation on the biosignal using a microcontroller; and
analyzing the biosignal based on the feature point.

9. A non-transitory computer-readable storage medium storing a program for controlling a computer to perform the method of claim 1.

10. An apparatus for monitoring a biosignal, the apparatus comprising:
a receiver configured to receive a biosignal and a reference signal;
a signal state determiner configured to determine a signal state of the biosignal based on the reference signal; and
a signal analyzer configured to analyze the biosignal using an analysis technique having a resource consumption depending on the signal state.

11. The apparatus of claim 10, wherein the signal analyzer comprises a microcontroller configured to perform a digital signal processing (DSP) operation on the biosignal to analyze the biosignal in response to the signal state being an unstable signal state.

12. The apparatus of claim 10, wherein the signal analyzer comprises:
a comparator configured to compare a characteristic of the biosignal with a threshold in response to the signal state being a stable signal state to determine whether the characteristic of the biosignal exceeds the threshold, the threshold being determined based on the biosignal or the reference signal;
a pulse generator configured to generate a pulse each time the comparator determines that the characteristic of the biosignal exceeds the threshold; and
a heart rate calculator configured to calculate a heart rate by measuring a pulse interval of pulses generated by the pulse generator, and calculating the heart rate based on the pulse interval.

13. The apparatus of claim 12, wherein the threshold is a threshold for an amplitude of the biosignal, or a threshold for a root mean square (RMS) of the biosignal, or a threshold for a feature point of the biosignal.

14. The apparatus of claim 10, wherein the receiver comprises a signal measurer configured to measure the biosignal and the reference signal.

15. The apparatus of claim 10, wherein the receiver comprises:
a signal measurer configured to measure the biosignal; and
a communicator configured to receive the reference signal from an external device configured to measure the reference signal.

16. The apparatus of claim 10, wherein the reference signal is an electrocardiogram (ECG) signal, or an accelerometer signal, or a half-cell potential (HCP) signal, or an impedance signal, or a lightwave signal, or a strain gauge signal, or a signal generated by a motion of a user.

17. The apparatus of claim 10, wherein the biosignal is an ECG signal, or an electroencephalogram (EEG) signal, or an electrooculogram (EOG) signal, or an electromyogram (EMG) signal.

18. The apparatus of claim 10, wherein the receiver comprises a signal measurer configured to be attached to a body of a user to measure the biosignal; and
the signal state of the biosignal is influenced by a motion state of a user or a location of the signal measurer on the body of the user.

19. A method of monitoring a biosignal, the method comprising:
selecting one of a plurality of analysis techniques having different resource consumptions based on a biosignal; and
analyzing the biosignal using the selected analysis technique.

20. The method of claim 19, wherein the plurality of analysis techniques respectively correspond to different stabilities of the biosignal.

21. The method of claim 19, wherein the selecting comprises selecting one of the plurality of analysis techniques having different resource consumptions based on a stability of the biosignal.

22. The method of claim 21, wherein a resource consumption of the selected analysis technique decreases as the stability of the biosignal increases.

23. The method claim 19, wherein the biosignal is a biosignal of a user, and is influenced by a motion of the user; and
the selecting comprises selecting one of the plurality of analysis techniques having different resource consumptions based on a characteristic of the biosignal indicative of the motion of the user.

24. The method of claim 23, wherein a resource consumption of the selected analysis technique decreases as the motion of the user decreases.

25. The method of claim 23, further comprising:
obtaining a reference signal indicative of the motion of the user; and
determining the characteristic of the biosignal based on the reference signal.
